(19) United States
(12) Patent Application Publication
(10) Pub. No.: US 2009/0203971 A1
(43) Pub. Date: Aug. 13, 2009

(54) System for Remote Monitoring of Physiological Parameters of an Individual, Method and Computer Program Product Therefor

(76) Inventors: Antonio Sciarappa, Torino (IT); Luigi Civera, Torino (IT); Giovanni Bestente, Torino (IT)

Correspondence Address: NIXON & VANDERHYE, PC, 901 NORTH GLEBE ROAD, 11TH FLOOR, ARLINGTON, VA, 22203, US

(21) Appl. No.: 11/922,027

(22) PCT Filed: Jun. 6, 2006
(86) PCT No.: PCT/EP2006/062931

§ 371 Date: Dec. 12, 2007

(30) Foreign Application Priority Data
Jun. 13, 2005 (IT) T02005A000407

(51) Int. Cl. A61B 5/00; G08B 23/00

(52) U.S. Cl. 600/301; 340/573.1

(57) ABSTRACT

System for the remote monitoring of physiological parameters of individuals, that includes:
a mobile module (11) to detect physiological parameters, that can be associated to an individual, including one or more sensors (24, 25, 32, 33, 34) to detect said physiological parameters and a wireless transceiver module (26) configured at least to transmit data relating to said physiological parameters over a wireless link (14);
a base station (12) configured to exchange data and controls over said wireless link (14) with said mobile module (11) to detect physiological parameters;
a monitoring centre (13) located remotely and configured to exchange data on at least one communication network (15) with said base station (12).

The mobile module (11) to detect physiological parameters includes a microprocessor control module (30) configured to process the physiological parameters and, in function of the results of said processing of physiological parameters, to command said transmission module (26) to operate between at least two operating conditions including:
a first normal operating condition that comprises operating transmission at constant intervals of said data relating to physiological parameters;
a second emergency operating condition, commanded by said microprocessor control module (30) should the result of said processing of physiological parameters identify a condition of alarm, comprising a continuous communication phase with said base station (12).

### FIELD OF THE INVENTION

The present invention relates to techniques for the remote monitoring of physiological parameters of individuals, in particular to remote monitoring of the elderly.

The invention was developed paying particular attention to its possible applications in monitoring elderly persons within old people's home or hospital settings.

For simplicity of illustration, in the remainder of the present description almost constant reference will be made to this possible field of application. It will however be understood that the scope of the invention is in fact general and thus not limited to this specific context of application, but also extends to include the monitoring of individuals at home, as well as to categories of persons such as the chronically ill or disabled.

As a general premise to the description of the known technology, of the problems underlying the invention and of the solutions proposed here, it appears useful to sum up some essential characteristics of the technical sphere within which the invention lies.

### DESCRIPTION OF THE KNOWN TECHNOLOGY

In the hospital setting, or in that of similar structures for the elderly such as old people's homes or sheltered accommodation, the need is felt to keep individuals under control in order to detect any situations of emergency in which the old person is unable to call for help autonomously. In this sphere, solutions are known which entail the use of remote control devices configured such as to be easy to activate by the old person with simple pressure, so as to be able to signal a condition of danger rapidly. However, these solutions have evident disadvantages in case of the onset of a sudden complete inability to move.

Solutions are likewise known that entail monitoring the individual's physiological parameters and giving the alarm if values associated to a condition of deterioration of the individual's organism are detected.

However, the effectiveness of these solutions is limited since the monitoring requires excessive power consumption, in particular due to the operations of reception and transmission by the module associated to the individual in order to detect the physiological parameters. Furthermore, the communication system used to transmit data and alarms to the control centre is not very flexible or adaptable, in particular when the system is used to control areas of large extension and in the presence of a number of individuals to be monitored.

### PURPOSE OF THE INVENTION AND BRIEF DESCRIPTION

The present invention has as its purpose that of providing a solution for monitoring the physiological parameters of an individual remotely such as to overcome the drawbacks inherent in solutions according to the known technology to which reference was made above.

According to the present invention, this purpose is achieved thanks to what is indicated in detail in the attached claims.

In particular, it will be appreciated that the present invention may be formulated in terms of process, in terms of system, and also in terms of a computer program product directly loadable into the memory of a digital computer and that is capable of perform the steps of a process according to the invention when the computer program is run on a digital computer.

As well as affording low consumption of the module to detect the physiological parameters, the proposed solution makes it possible to install, in a simple manner, a system then guarantees efficient communications, including when it operates in the presence of movements of a number of individuals over a large area.

### BRIEF DESCRIPTION OF THE ATTACHED DRAWINGS

The invention will now be described, as a simple example without limiting intent, with reference to the attached drawings, in which:

FIG. 1 represents a system architecture according to the invention;

FIGS. 2a, 2b and 2c represent three different views of a module operating in the system according to the invention;

FIGS. 3a and 3b represent, in diagram form, the signals exchanged in the system according to the invention.

### DETAILED DESCRIPTION OF SOME EMBODIMENTS OF THE INVENTION

The proposed procedure and system are substantially based on the use of a mobile module for the detection of physiological parameters, preferably in the form of a wrist-watch so as to be little intrusive, able to detect certain physiological parameters such as temperature, movement/immobility, cardiac activity, and transmit them via radio to a base station connected at medium range (12-20 metres), that interconnects with the public network or the in-house network and transmits the information to a remote control centre.

The mobile detection module is configured so as to be activated only in an emergency operating condition, overriding a standard operating condition that entails transmission at constant intervals. According to a further aspect of the invention, the system is fitted with a telecommunications architecture that entails a form of roaming management, that is the possibility to transfer the communication with the mobile module from a base station associated to an area to a base station that controls another area within environments such as hospitals or old people's homes in which the system operates. This roaming function, as will be described in more detail, is active both in normal operating conditions and in emergency conditions, so that it is possible to search for other base stations to receive the alarm should the associated base station not be available.

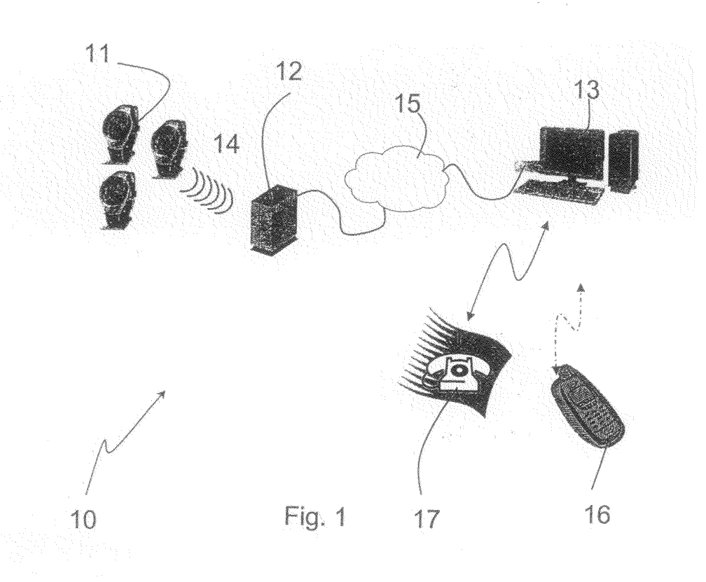

FIG. 1 shows in diagram form a system for the remote monitoring of physiological parameters of an individual according to the invention, indicated as a whole with reference 10.

Reference 11 indicates a plurality of modules to detect the physiological parameters in the form of a wearable wrist-watch that communicate through a wireless or radio link, 14, with a base station 12 for data collection. This base station 12 communicates by means of a telecommunications network 15, that may be a telephone network or a network of the IP (Internet Protocol) type with a remote control centre 13 that is capable of carrying out a monitoring application routine to process the data and if required to send alarms to mobile telephones 16 or to fixed telephones 17 or to activate sound or luminous warning signals.

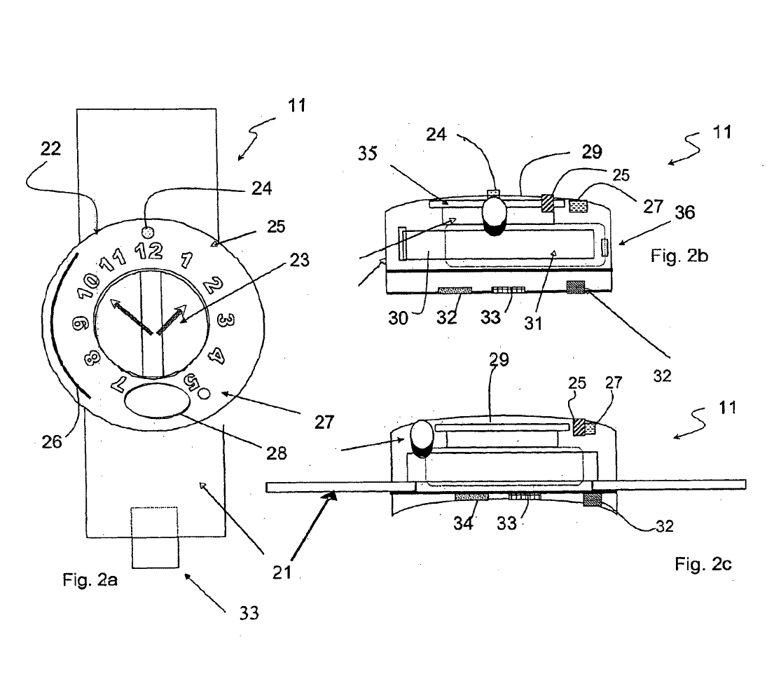

The module to detect physiological parameters 11 is shown in FIG. 2a in plan where 21 indicates a strap, which bears a case 22 on which are located an analog watch 23 or alternatively a photograph/image, a photodiode 24, or also a photoresistor, to verify external environmental conditions, an external temperature sensor 25, a strip antenna 26, a LED diode 27, as well as a call button 28.

FIG. 2b shows a front view of the mobile module 11, in which the photodiode 24 can clearly be seen to be situated on a glass 35 to protect the analog watch 23, below which is placed a printed circuit containing the processing module 30 associated with a power supply battery 31. In the lower part of the mobile module 11 is also situated a body temperature sensor 32. The mobile module 11 also includes a reset button 36.

The disposition of the components of the mobile module 11 can also be seen in FIG. 2c, which shows the mobile module 11 in diagram form in side view.

Furthermore, as FIG. 2b shows, the mobile module 11 also includes:
an accelerometer 34, acting as a movement sensor, positioned on the printed circuit;
a piezoelectric sensor 33 to detect cardiac activity, located on the strap 21.

The mobile module 11 possesses firmware features lodged in the processing module 30 that provide:
a consumption management routine that maintains the system fully active from the standpoint of receiving and transmitting only in emergency situations, to optimise battery life;
wireless data transmission through the wireless link 14, using the 2.4 GHz ISM band, and with possibility of two-way communication;
pre-processing of the acquired data (management of masks, thresholds, correlations);
remote management of the configuration (download firmware, parameters, etc.).

The mobile module 11 is configured as a wearable wrist-watch according to ergonomic criteria functional to the field of application of the system.

The dimensions and shape of the watch are in line with those of normal watches on the market, not excessively noticeable, for better acceptability by the elderly.

The strap 21 is very simple to fasten and unfasten and can easily be adjusted: for this purpose a preferred version has a fastening employing “Velcro”™.

The chosen materials are soft to adhere to the person's wrist, also taking into consideration the fragile skin of the elderly, that can easily be grazed. The chosen materials are also resistant to knocks and water, while the messages and labels are in Italian avoiding terms in foreign languages such as ON/OFF, ALARM, . . . to aid understanding by the elderly.

The analog watch 23 is preferred over watches with digital indications again to aid understanding by the elderly, as observation has shown that the elderly use watches with analog dial. In the same way a command is provided to adjust the time, through a crown wheel or button, following the conventions in use on watches, on the right hand side of the analogue watch 23.

The call button 28 is made in a different shape and size than the command to adjust the time. This call button 28, activated at need by the individual, is located on the glass 29 close to the periphery of an upper face of the mobile module 11, below the dial of the watch 23, in a position that protects it from involuntary activation and at the same time is easy to find and convenient to press.

Coming to the base station 12 for data collection, it represents the control unit inserted in the home/living space (also known as RSA) of the user. This base station 12 collects data sent by the mobile module 11 and transmits them directly to the remote service centre 13 through the Internet network, which thus forms the telecommunications network 15.

The base station 12 manages the various information coming from different mobile modules 11 that operate as collection stations and provides an initial “diagnosis”/interpretation in real time for the operator who, depending on the service protocol activated and the severity of the alarm, will activate the appropriate remote assistance procedures.

In detail, the base station 12 for data collection has the following features:
connection to the telecommunications network 15, telephone line and, in general the IP network;
integrated radio frequency reception unit on 2.4 GHz ISM band;
possibility of local processing for an initial processing of data transmitted/received from the mobile module 11 or from other peripherals present in the home.

From the architectural standpoint the base station 12 comprises a ColdFire 5272 UCdimm™ processor module, managed by a Linux operating system for embedded systems (uCLinux). The base station 12 comprises a concentration node that enables the mobile modules 11 to send information to the service centre 13. The software needed to manage the base station 12 is configured to perform the following operations:
management of a Transceiver CC2400 transceiver;
management of the communication protocol;
management of synchronisation of information with other base stations with which the station 12 is connected;
management of communication with the service centre;
information processing;
integration with possible alarm devices if alarms are detected.

The communication protocol implemented by the base station 12 will now be described.

The main objective of this protocol is to enable communication between the base station 12 and the mobile modules 11.

The architecture must permit the creation of a network of base stations capable of ensuring wireless cover of an entire building. The mobile modules 11 must be able to communicate with the nearest base station 12. The main features of the protocol are that it manages a number of mobile modules 11 connected to a single base station 12, handles radio interference, manages a signalling plan and transmits information.

For this purpose, as mentioned the base station 12 preferably comprises a ColdFire 5272 UCdimm™ processor module (with ColdFire MCF5272 Motorola microcontroller, serial interface, Ethernet and modem), managed by a Linux operating system for embedded systems (uCLinux), as well as an RF CC2400 transceiver.

The mobile module 11 likewise includes in the processing module 30 an RF CC2400 transceiver, as well as a Silicon Laboratories C8051F311 microcontroller that implements a proprietary operating system.

The main technical characteristics of the CC2400 transceiver used for radio transmission are: transmission band: 2.4-2.4835 GHz (unlicensed ISM band); data rates: 10 kbps, 250 kbps and 1 Mbps, with programmable output power, base band programmable modem, packet management hardware, data buffering features and digital RSSI output.

The communications protocol is designed, as well as for the features described above, to optimise power consumption and the use of memory on the mobile module 11 side.

In this connection, since the transceiver consumes a significant quantity of power in relation to the batteries that can be used (batteries of small size), the communication protocol on the mobile module 11 side is organised so as to keep the transceiver on for as short a time as possible and activate it in case of emergency.

To sum up, the chief features that the protocol must implement are:
on the mobile module 11 side:
   search for the closest base station 12 (if possible by using an access technique to a signalling channel in the link 14 of the combined type: FDMA and TDMA);
open the connection;
transmit data packets (subdividing long packets);
manage signals acknowledging reception and re-transmit non-received packets;
manage a transmission with high bit-rate to send information in case of emergency;
close the communication;
on the base station 12 side:
manage the signalling channel (TDMA-FDMA);
assign a communication channel in the wireless link 14 (the signalling plan must assign a frequency free for communication between the mobile module 11 and the base station 12, in case of the use of a channel access technique of the FDMA type);
data transmission/reception from a number of mobile modules 11 (data from all the mobile modules 11 connected to the base station 12 must be acquired), reception buffer management, reassembling of packets;
release of communication channel (the frequency must be made available for new communications);
management of a transmission with high bit-rate to send information in case of emergency;
management of the cumulative ACKNOWLEDGE mechanisms.

The requirements linked to the transmission of data relating to the physiological parameters acquired by the sensors, as mentioned above, entail the definition of two operating conditions of the mobile module 11:
a standard operating condition associated to a data monitoring phase;
an emergency operating condition associated to an alarm phase, initiated by the processing module 30 that makes a pre-analysis of the data acquired by the sensors based on the values detected and appropriately correlated; detection of a situation of possible anomaly activates the “awakening” of the transmitting apparatus enabling the data to be sent to the remote control centre 14.

The processing module 30 includes a sensor sampling module. In the standard operating condition, when such sampling module has accumulated sufficient data it activates the transceiver, so as to put the mobile module 11 into listening mode on a signalling or common channel awaiting an identification signal from the base station, BS_ID, provided by a base station 12 of the network associated to the area in which the mobile module 11 finds itself. A contention phase now begins regulated by a system with random and priority back-offs (to guarantee mobile modules 11 that have already lost previous contentions a higher probability of success). Within the field of minimum and maximum wait for a priority level, discrete intervals are determined regulated on the maximum propagation round-trip time that can come about in the system. A REQ request signal may only be sent at the beginning of one of these intervals, so that there are only collisions between REQ request signals from different mobile modules 11, but not between REQ request signals and ACK acknowledge signals from the base station 12, so as to react rapidly to a collision. The mobile module 11 that receives the ACK acknowledge signal from the base station 12 positions itself on a frequency that is communicated to it and of which at that moment it will have exclusive use, and may send the data in a time slot that is guaranteed to it. At the end the mobile module 11 returns to a rest condition, also known as sleep mode, and the base station 12 will launch a new signal identifying the base station, BS_ID.

In the emergency operating condition, as soon as the mobile module 11, processing the data received from the sensors, detects an alarm situation, the radio part is activated; on reception of an identification signal BD_ID from the base station a request frame, or packet, is sent that specifies the alarm situation. This request frame is sent at a time instant situated before the minimum possible time to send every other type of request, guaranteeing that the request frame will surely win the contention phase mentioned above. At this point the base station 12 associates to itself the mobile module 11 in alarm and starts a polling phase, or invitation to transmit, in which communication is continuous and the sensors of the mobile module 11 sample the physiological parameters of the individual at a higher frequency to operate real-time monitoring. Polling is periodically suspended to enable the base station 12 to send on the signalling channel a signal BS_ID identifying the base station in order to detect the possible presence of other devices in alarm. In the frame associated to this further identification signal it is specified that there is an emergency underway so that requests from mobile modules 11 not in alarm become less frequent so that the radio module will remain in sleep mode for as long as possible, avoiding power wastage.

During the polling phase systems are implemented to maintain the mobility of the mobile module 11 in alarm, and thus the association with other base stations 12, in other words to manage the roaming. If the mobile module 11 does not receive requests from the base station 12 within a limited time, or timeout, it considers itself to be dissociated from it, and launches the alarm towards the first base station that makes itself available through the base station identification signal BS_ID. Similarly, if the base station 12 cannot contact the mobile module 11 in alarm for a set number of times, it considers that mobile module 11 to be dissociated and discontinues polling with regard to it. However, the application level that operates in the remote control centre 14 is advised of this situation to avoid an alarm being considered terminated that is in reality still under way.

The format of the data packet may be configured depending on the specific needs and comprises the following fields:
preamble;
synchroniser word;
data;
error correction code.

To construct the data packet a so-called buffered mode may be used in transmission that entails:
adding a programmable number of preamble bytes, for example 32 bit;
adding the synchroniser word, for example 16 or 32 bit;
calculating and adding the error correction code relative to the data field.

The data field may be of 8×n bit, whereas the error correction code is of 16 bit.

In reception a packet handling procedure is used to analyse the packet received and verify its validity by:
detecting the synchronism word:
calculating and checking the error correction code received.

The packet handling procedure may be used, optionally, in combination with the coding 8B/10B, which will be applied exclusively to the data field and to the error correction code.

The remote control centre 14, processing the data, enables alarms or messages to be generated in one or more of the following cases:
if the mobile module 11 detects a sudden change in the acceleration value, followed by a period of inactivity that exceeds a certain threshold time, correlated to a slow decrease in skin temperature and heart rate;
if the mobile module 11 detects an increase in skin temperature and heart rate for long periods;
if it detects an increased environmental temperature and decreased physical activity of the user;
if it detects a halt in the heart beat and a decrease in skin temperature.

The cardiac activity signal processing procedure is determined on the basis of a study carried out under the dual profile of performance in clinical terms and the compatibility with the technical requirements of the system, with regard to both the acquisition system characteristics and the calculation and transmission resources available on the watch.

The primary goal of this cardiac activity signal processing procedure is to determine the heart beat by processing the signals acquired by the piezoelectric sensor. Local processing on the mobile module 11, considering the restrictions set by the system in terms of processing capacity, chiefly consists in procedures based on threshold detection or thresholding techniques, which entail placing as zero signals below a certain threshold and spectral estimation techniques (techniques based on Fourier analysis and time-frequency transform), in order to detect the peaks corresponding to the beats.

Alarms can be programmed in function of the user's requirements, habits and needs and those of the sheltered housing.

The system and procedure described here thus enable physiological parameters to be advantageously detected through a mobile module associated in an ergonomic fashion to the wrist of the individual to be monitored. This mobile module advantageously operates in at least two configurations, including a standard and an emergency configuration, basing the transition from one to the other on a pre-analysis of the data detected by the module sensors. This enables consumption to be reduced and makes use of the module practical.

Furthermore, advantageously, the module is interfaced with one or more wireless base stations through a protocol that permits both the effective management of priorities among a number of appliances and thus of multi-user situations, and the adoption of a roaming function, so that it becomes simple to follow an individual in movements over large areas, for example hospital buildings, enabling the mobile module for detecting physiological parameters to be associated to differently-located base stations at different times. Note that this also makes it possible, within certain limits, to follow the movements of the individual wearing the mobile module.

According to another aspect of the invention, advantageously, thresholding and spectral analysis techniques are adopted with regard to the cardiac signal detected by the sensors.

Of course, without prejudice to the principles of the invention, the construction details and embodiments may be widely varied with regard to what is described and illustrated here without thereby departing from the scope of the invention. In this connection, it is underlined again that, although for the sake of simplicity of illustration in the present description almost constant reference has been made to the possible application of the invention to one context, the scope of the invention is in fact general and thus not limited to that specific application context.

1. System for the remote monitoring of physiological parameters of an individual, that includes:
at least one mobile module (11) to detect physiological parameters, that can be associated to at least one individual, including one or more sensors (24, 25, 32, 33, 34) to detect said physiological parameters and a wireless transceiver module (26) configured at least to transmit data relating to said physiological parameters over a wireless link (14);
at least one base station (12) configured to exchange data and controls over said wireless link (14) with said mobile module (11) to detect physiological parameters;
a monitoring centre (13) located remotely and configured to exchange data over at least one communication network (15) with said base station (12)
characterised in that
said mobile module (11) to detect physiological parameters includes a microprocessor control module (30) configured to process the physiological parameters and as a function of the results of said processing of the physiological parameters to command said transmission module (26) to operate between at least two operating conditions including:
a first normal operating condition that comprises operating the transmission of said data relating to physiological parameters at constant intervals;
a second emergency operating condition, commanded by said microprocessor control module (30) when the results of said processing of the physiological parameters identifies a condition of alarm, comprising a continuous communication phase with said base station (12).

2. System according to claim 1, characterised in that said at least one mobile module (11) and said at least one base station (12) are configured to co-operate according to a protocol for mobile telecommunications networks that includes a roaming management routine and a multi-user management routine.

3. System according to claim 1, characterised in that said mobile module (11) is configured to perform transceiver functions of the mobile type, said transceiver functions of the mobile type including one of more of the following operations:
search for the closest base station (12), in particular through channel access techniques of the combined FDMA and TDMA type;
open a connection over the wireless link (14);
transmit data packets, subdividing long packets;
manage signals acknowledging reception (ACK) and re-transmit non-received packets;
manage a transmission at higher bit-rate than a bit-rate in normal conditions to send information in emergency conditions;
close the connection on the wireless link (14).

4. System according to claim 1, characterised in that said base station (12) is configured to perform one or more of the following operations:
manage a signalling channel, in particular in TDMA or FDMA mode;
assign a communication channel on the wireless link;
exchange data with one of more mobile modules (11) associated to said base station (12);
release said communication channel;
manage a transmission at high bit-rate to send information in emergency conditions.

5. System according to claim 1, characterised in that in said first normal operating condition, said transmission at constant intervals entails placing said module (26) in a listening mode on a common signalling channel belonging to said wireless link (14) to await an identification signal (BS_ID) distinctive of the base station (12).

6. System according to claim 1, characterised in that said base station (12) is configured to manage a contention phase among a number of mobile modules (11).

7. System according to claim 6, characterised in that said contention phase includes an operation to discretise intervals to send a request signal (REQ), said discrete intervals being in particular regulated on the system's maximum propagation round-trip time, that may occur in the system.

8. System according to claim 6, characterised in that said mobile module (11) is also configured such that, on reception of an acknowledge signal (ACK) from the base station (12), it commands said transceiver module (26) to operate in exclusive use on a frequency in said wireless link (14) communicated by said base station (12) and to send data within a guaranteed interval of time.

9. System according to claim 1, characterised in that said module (11) is configured, in said emergency operating condition, to activate said transceiver module (26) and to send, on reception of an identification signal from the base station (BS_ID) a request packet indicating the alarm, at a moment in time preceding to a minimum possible time to send the request (REQ) in the normal condition in order to guarantee that the request frame will surely win the contention phase.

10. System according to claim 9, characterised in that, in said emergency operating condition said base station (12) is configured to associate to itself the mobile module (11) in alarm and to start a polling phase in continuous communication of the data transmitted by said mobile module (11), in said polling phase said one or more sensors (24, 25, 32, 33, 34) of the mobile module (11) sampling the physiological parameters at a higher frequency compared to a frequency used in said normal operating condition.

11. System according to claim 10, characterised in that said polling phase in continuous communication is periodically suspended to enable the base station (12) to send on a common channel of the wireless link (14) a further signal identifying the base station (BS_ID) in order to detect the possible presence of other mobile modules (11) in emergency condition.

12. System according to claim 11, characterised in that it provides for the association to said further signal identifying the base station (BS_ID) of information relating to the presence of a mobile module (11) in emergency condition and that said mobile modules (11) are configured to increase the interval of time to place said mobile module (11) in listening mode on the common channel of the wireless link (14) awaiting an identification signal (BS_ID) distinctive of the base station (12) in order to limit power consumption.

13. System according to claim 10, characterised in that, during the polling phase, in order to maintain the mobility of the mobile modules (11) in alarm said roaming routine implements one or more of the following operations:
dissociate the mobile module (11) from the base station (12) if said mobile module (11) does not receive requests from said base station (12) within a time limit and send said request packet indicating the alarm to a base station (12) that makes itself available through the signal identifying the base station (BS_ID);
dissociate the base station (12) from the mobile module (11) if said base station (12) is unable to contact the mobile module (11) in alarm for a set number of attempts, halt the polling phase and send an alarm maintenance signal to the remote control centre (13).

14. System according to claim 1, characterised in that said one or more sensors (24, 25, 32, 33, 34) comprise one or more from among the following sensors:
an external temperature sensor (25);
a photodiode (24) to check the condition of the external environment;
a body temperature sensor (32);
a movement sensor (34), in particular a two-axis accelerometer;
a sensor to detect cardiac activity (33), in particular a piezoelectric sensor.

15. System according to claim 1, characterised in that said microprocessor control module (30) includes a module to make a pre-analysis of the data acquired by said one or more sensors (24, 25, 32, 33, 34) and a sampling module of said one or more sensors (24, 25, 32, 33, 34).

16. System according to claim 1, characterised in that said module (11) is configured in the form of a wearable wrist-watch.

17. System according to claim 1, characterised in that said control centre (14) is configured to process data transmitted by one or more base stations (12) and generate alarms or messages in one or more of the following cases:
if the mobile module (11) detects a sudden change in the acceleration value, followed by a period of inactivity that exceeds a certain threshold time, correlated to a slow decrease in skin temperature and heart rate;
if the module (11) detects an increase in skin temperature and heart rate for long periods;
if the module (11) detects an increased environmental temperature and decreased physical activity of the user;
if the mobile module (11) detects a stop in the heart beat and a decrease in skin temperature.

18. System according to claim 1, characterised in that it includes processing the cardiac activity signal by applying to a cardiac signal detected by the sensor (34) threshold detection techniques that involve placing at zero signal values detected by the sensor (34) below a prefixed threshold and/or spectral estimation techniques, in order to detect peaks corresponding to heart-beats.

19. System according to claim 1, characterised in that said module (11) includes a call button (28) that can be activated in case of need by the person to send a further alarm.

20. System according to claim 1, characterised in that said telecommunications network (15) is a telephone telecommunications network and/or of the Internet Protocol type.

21. Process for the remote monitoring of physiological parameters of individuals including the operations performed by the monitoring system according to claim 1.

22. Computer programme product directly loadable into the memory of an electronic digital computer and including software code portions to perform the steps of the process according to claim 21.

23. Computer programme product directly loadable into the memory of an electronic digital computer and including software code portions to perform the steps of the process according to the operations of the monitoring system according to claim 1 when the computer programme product is executed on a computer.
